(19) United States
(12) Patent Application Publication
(10) Pub. No.: US 2022/0023672 A1
(43) Pub. Date: Jan. 27, 2022

(54) Coating Compositions

(71) Applicant: THEUNSEEN LIMITED, London (GB)

(72) Inventors: Lauren Bowker, London (GB); Louise Anderson, London (GB)

(21) Appl. No.: 17/297,563

(22) PCT Filed: Nov. 27, 2019
(86) PCT No.: PCT/EP2019/082703

§ 371 Date: May 27, 2021

(30) Foreign Application Priority Data
Nov. 27, 2018 (GB) 1819324.3

(51) Int. Cl. A61Q 3/02; A61K 8/02; A61K 8/04; A61K 8/55; A61K 8/81

(52) U.S. Cl. CPC A61Q 3/02; A61K 8/0295; A61K 8/042; A61K 8/55; A61K 2800/95; A61K 8/817; A61K 2800/438; A61K 2800/591; A61K 2800/805; A61K 8/8152

(57) ABSTRACT

A coating composition for coating nails to provide an aesthetic colour-change effect using a liquid crystal. The composition is a gel which is spreadable and contains a curable oligomer and/or, a monomer, a curing initiator and an unsealed liquid crystal.

This invention relates to a coating composition, particularly to a liquid crystal gel composition for coating a substrate, particularly nails, artificial nails and existing coatings on nails. The invention is particularly concerned with a gel comprising a liquid crystal component which provides a colour-change effect.

A wide range of decorative cosmetics for coating substrates such as keratinous living substrates, for example nails, skin, lips and the like and non-living substrates such as artificial nails, for example acrylic nails and nail extensions are available in the personal care market to provide aesthetic and/or functional effects. Such compositions are typically solvent-based or may be aqueous-based depending on the particular type of product and typically must comply with toxicology and other safety and testing standards and protocols.

Commercially formulated nail products are typically in liquid or gel form and comprise a number of components to provide a desirable combination of characteristics such as ease of application and evenness of coating, durability and resistance to chipping and washing, vibrant colour, reasonable drying time and non toxic. Nail coatings typically comprise organic solvents, a colour pigment and a film forming component and upon deposition, the solvents evaporate to leave a dried durable coating and may include rheology modifiers, photoinitiators, plasticisers, colour additives, for example pearl agents, mica and the like, viscosity modifiers, UV additives and other components.

Nail compositions having a chromic component providing a colour change effect are known. Chromic components change between a first and second state which may both be coloured or white or one coloured or white and the other colourless, in response to a change in an external condition such as application of energy for example through a change in temperature, magnetics or pressure or incidence of light. Chromic components which change colour with a change in temperature are known as thermochromic, with pressure as piezochromic, with water, hydrochromic and, with light, as photochromic. Chromic components which change colour or opacity between different states or in response to a stimulus are referred to herein as “colourants”. Colour change in chromic compositions may be provided by different types of materials including leuco-colourants and liquid crystals.

Thermochromic compositions reversibly change colour or change between different coloured states or a coloured and colourless state when subjected to a change in temperature of a sufficient magnitude. Typically, a change in temperature will lead to a change in colour of the colour-change material. With an increase or decrease in temperature, the colour-change material will typically retain colour until a maximum or minimum temperature for retention of the complete coloured state is reached. The composition will then progressively change colour or become colourless as the temperature increases or decreases until it has completely changed colour. As the thermochromic composition cools it will reach a temperature below which the colour changes disappears or reappears. Thermochromic materials, having standard colour change schemes and bandwidths, that is a difference in temperature at which colour change occurs from a first to a second colour and in reverse from the second colour to the first, are commercially available, for example from LCR Hallcrest.

Commercially available nail coating products are typically based on organic solvents. Aqueous-based nail coating compositions are known but commercialisation has proved difficult.

In chromic cosmetic products, colour-change may be provided by a leuco dye although certain solvents may affect the colour-change properties of such materials and careful formulation is required. The colour-change effect and the conditions under which it occurs, for example a particular temperature range may be controlled by modifying the leuco dye composition in a manner known to those skilled in that field.

We have now found that a colour-change nail product having intense colour without the liquid crystal being impaired by providing a composition which has a liquid crystal and a curable oligomer and/or monomer which forms a gel upon curing.

In a first aspect the invention provides a coating composition comprising a chromic component comprising a liquid crystal, and a curable oligomer and/or, a monomer and a curing initiator.

The coating composition is suitable for application to natural nails and artificial nails. Preferably, the coating product is a gel comprising a chromic component comprising a liquid crystal, and a curable oligomer and/or, a monomer and a curing initiator.

Liquid crystal, particularly encapsulated liquid crystal, is typically not stable in organic solvent. The coating composition may be substantially free of organic solvent.

The coating composition may comprise an oligomer and upon curing provide a gel coat or may comprise a monomer and upon curing provide a gel coat or a non-gel coat.

In a second aspect, the invention provides a cured coating comprising a chromic component comprising a liquid crystal and a continuous polymer phase which is derived from a cured oligomer and/or, a cured monomer, and an initiator, wherein the chromic component is dispersed in the said continuous phase.

In a third aspect, the invention provides a method of providing a cured coating which comprises applying a coating composition to a substrate to be coated which coating composition comprises a chromic component comprising a liquid crystal, and a curable oligomer and/or a monomer and an initiator, curing the curable oligomer and/or monomer to form a polymer, preferably by irradiating the applied coating composition, to provide a continuous phase comprising the polymer and a disperse phase comprising the chromic composition dispersed in the continuous phase.

The coating is especially suitable for coating keratinous material directly or indirectly, particularly nails and for coating artificial, non-keratinous nails and nail extensions, for example acrylic nails. The coating may be applied to an existing coating, for example a base coat, a gel coat, a shellac coat and polished nails and to uncoated nails.

The term “gel” as employed herein refers to a solid, colloid having a disperse phase, for example a solid disperse phase, and a continuous phase, for example a liquid phase which is flowable. The liquid phase may be sufficiently viscous to behave like a solid to. The disperse phase suitably comprises liquid crystal and the continuous phase comprises the oligomer and/or monomer which is curable provide a polymer matrix with islands or regions of the dispersed liquid crystal. The gel may be visibly elastic or jellylike, for example such as gelatin, or relatively solid and rigid, for example such as silica gel.

The coating may be tailored, to provide a desired aesthetic appearance for particular circumstances or moods and adapted to provide a faster or slower colour change with single or multiple colour change within a bandwidth of temperatures or other external parameters. For example, the coating may be layered or mixed with other non colour change or colour change, coloured gels with the same formulation. The colour change may be reversible or irreversible.

Advantageously, the product of the invention provides desirable qualities including one or more of rapid speed of cure, hardness, scratch resistance, adhesion to the underlying nail or undercoat, UV stability, flexibility, ease of removal with known solvents such as acetone, a tailored finish, for example shiny or matt and minimal or no shrinkage so as to avoid causing damage to the nail.

The term “chromic component” refers to a material which may undergo one or more successive colour changes. The colour change may be reversible or irreversible. The colour change may be in response to the application, presence or change of a stimulus, for example heat, light (photochromic), pressure, hydrochromic colourants which are responsive to contact with water, vapochromic colourants, which are responsive to the presence of certain gases or vapours and may be used to detect the presence of pollution or other environmental agents, solvatochromic colourants which respond to changes in polarity, ionochromic colourants which respond to changes in ion concentration, halochromic colourants which can detect changes in pH, for example changes in the pH of the substrate of the nail or the adjoining skin.

The chromic component may change colour over time without any other stimulus whether in seconds or days, for example by tailoring the bandwidth such that component is in a particular state presenting a different colour for a shorter or longer period of time with or without a particular stimulus.

The liquid crystal may be unsealed or encapsulated. Preferably the liquid crystal is unsealed. The unsealed liquid crystal preferably comprises a polymer-dispersed liquid crystal. A polymer-dispersed liquid crystal comprises a discrete phase comprising the liquid crystal dispersed in a continuous polymer phase in which the liquid crystal is present in droplets, typically of a micron scale, for example from 0.1 to 100 microns.

The liquid crystal suitably provides a colour-change effect according to changes in ambient or applied external conditions, for example temperature change and light. Suitable liquid crystals include cholesteric liquid crystals which are typically based on or comprise cholesterol or other sterol derived compounds including cholesteryl carbonate esters. Other suitable liquid crystals include non-sterol based compounds, for example phenyl benzoate esters. Suitable liquid crystals are available from a range of sources including LCR Hallcrest.

In a preferred embodiment, the liquid crystal comprises a liquid crystal which is immiscible or poorly miscible with the oligomer and/or monomer. For example, where the oligomer and/or monomer is highly polar or comprises polar substituents, the liquid crystal preferably comprises cholesteric liquid crystal. In other embodiments, non-sterol based liquid crystals, for example phenyl benzoates, are preferred as they provide more intense or bright colours offering an enhanced aesthetic effect and also allowing a lower level of liquid crystal to achieve a given brightness or intensity as compared to a cholesteric liquid crystals.

The liquid crystal may comprise an encapsulated liquid crystal in addition to or instead of the unsealed liquid crystal. Suitably, the encapsulated liquid crystal is incorporated with a minimal amount and preferably no water.

The encapsulate suitably comprises a known encapsulation material which is compatible with the liquid crystal. In one embodiment, the liquid crystal chromic component is suitably encapsulated in a wall comprising a gelatin and gum arabic mixture, for example gum arabic and/or a gluteraldehyde cross-linked gelatin. Other encapsulate materials which are known or subsequently devised may be employed. Examples of other types of capsules include siloxane based capsules, thermoplastic polymer based capsules and thermoset polymer based capsules.

Suitable liquid crystals include cholesteric liquid crystals which are typically based on or comprise cholesterol or other sterol derived compounds including cholesteryl carbonate esters. Other suitable liquid crystals include non-sterol based compounds, for example phenyl benzoate esters. Suitable encapsulated liquid crystals are available from a range of sources including LCR Hallcrest. Non-cholesterol based liquid crystals such as phenyl benzoate esters provide bright, strong colour effects with high reflectance, suitable for premium products. Cholesteric-type liquid crystals typically provide colour effects which are less intense than non-cholesteric liquid crystals but are of lower cost and may be suited to larger volume markets. A mixture of non-cholesteric and cholesteric liquid crystals may be employed to optimise product performance and cost. In a preferred embodiment, the liquid crystal suitably comprises only non-cholesteric liquid crystals.

Where the colourant is encapsulated, the encapsulation suitably comprises a known encapsulation material for example a gelatin and gum arabic mixture se. The wall material preferably comprises from 1 to 30%, more preferably 5 to 25%, for example 8% or 20% by weight of wall material based on the weight of the encapsulated liquid crystal.

The liquid crystal may be at any desired level in the coating composition from 1 to 99%. At lower levels, the liquid crystal will be employed at a level to provide the desired concentration and effect. The liquid crystal may be provided at higher levels provided the oligomer and/or monomer are present at a level to provide a continuous phase which adheres to the intended surface. The coating is suitably present at a level of 10 to 90%, preferably 30 to 80%, more preferably 35 to 75%, especially 40 to 70% by weight.

The chromic component may be selected so as to provide a single colour above a pre-determined temperature or below a pre-determined temperature.

The chromic component may be tailored to provide a colour-change after a period of time has elapsed rather than in response to a particular stimulus.

The initiator may be selected from any known initiators for the oligomer or monomer component employed. Preferably the initiator is selected from a thermally activated initiator and a photoinitiator. Examples of suitable photoinitiators include ethyl-4-(dimethylamino)benzoate, isoamyl 4-(Dimethylamino)benzoate, IRGACURE 2959 and ethyl (2,4,6-trimethylbenzoyl) phenyl phosphinate. An example of a suitable thermal initiator includes 4,4′-Azobis(4-cyanopentanoic acid.

The coating composition may also comprise a colourant. The colourant may comprise any material which presents a single colour in the conditions in which the composition is to be is used. A single colour may be provided by a colourant which does not change colour, for example a dye and/or a pigment or by a liquid colourant which is capable of colour change for example a liquid crystal or leuco colourant, but which colour change does not occur under the conditions of normal usage of the composition, known as a “static” colourant. The term “static” refers to a leuco colourant or a liquid crystal which does not change colour under the conditions of use. Thus while the colourant is capable of colour-change, it does not typically exhibit colour change under the conditions of use and shows a fixed colour.

Preferably, the colourant is selected from a static liquid crystal, a dye and a pigment in solid, preferably powder form.

Suitably, the colourant comprises a dye. Pigments may scatter light providing a “whitening” effect or haziness. A dye and/or pigment may accordingly be selected according to the intended desired effect. A pigment may provide a more singular colour change.

The term “singular” refers to a “two-tone” colour system, either a static colour or a marked colour change from colourless to coloured, or coloured to colourless, or from a first colour to a second colour, or a fluctuating colour between two colours as opposed to the traditional spectrum of colours, red, green and blue and transition between them typically observed with liquid crystal. For example a singular liquid crystal system may fluctuate between two colours such as green and blue without showing red. The term “singular” also refers to a single colour being observed above or below a certain pre-determined or tailored temperature. A singular colour liquid crystal suitably has a thicker encapsulate wall, for example 5 to 10 microns.

Haziness may be imparted intentionally to provide a visual effect by use of a pigment or by applying multiple layers of the liquid crystal or multiple layers of the continuous phase such that reflected light is diffracted.

The coating composition may comprise the colourant and colour-change component as a single composition in which the colourant and colour-change component are intimately mixed or may be applied to the nail as an undercoat prior to application of the coating composition.

The colourant is a fixed colour and enables or enhances visual appearance or enhancement of the properties of the liquid crystal. The colourant may be any colour for example red, orange, yellow, green, blue, indigo, violet, brown and black. Darker colours are preferred as they provide a more intense colour effect to the observer. Lighter colours may offer a pearlescent or opalescent effect which may be desirable in some cases.

The colourant may be incorporated into coating composition as a powder at any desired level.

The colourant may be black or blue, for example carbon black, In a preferred embodiment, the colourant comprises carbon black, iron oxide, mica, graphite synthetic melanin and polydopamine pigment. Examples of suitable black dye or pigments include products available under the trade name MINISO.

In a further aspect, the invention provides a coating composition comprising a chromic component comprising an unsealed liquid crystal, a curable oligomer and/or a monomer and an initiator and a black colourant, for example a black dye or a black pigment.

Advantageously, a coating composition, especially a gel coating composition, comprising a black dye or pigment in a single composition is easier to use than applying separate compositions containing a dye or pigment and a liquid crystal. Application in separate coats requires the first coat to cure or dry before application of the second, overlying coat. In a coating composition including a dye or pigment, a proportion of the dye or pigment may overlie liquid crystal and thereby reduce the effective reflectance of the liquid crystal. In this embodiment, a greater level of liquid crystal may be employed to mitigate or avoid any reduction in brightness due to the presence of black dye or pigment in the composition.

The curable oligomer suitably provides a polymer film upon curing. Preferably, the oligomer is polar and immiscible with the liquid crystal. Upon curing, the curable oligomer forms a film as a continuous phase with small dispersed liquid crystal regions within the continuous phase. The coated composition provides an attractive aesthetic effect, in particular excellent surface reflection, providing intense colours, contrast between different colours and responsive colour change and adherence to the substrate and other desirable characteristics may be secured.

The curable oligomer and/or monomer may be selected such that the liquid crystal and curable oligomer and/or monomer are immiscible.

In another embodiment, the oligomer and/or monomer may be selected such that the liquid crystal and oligomer and/or monomer are miscible. Upon curing the oligomer and/or monomer, the liquid crystal component suitably separates from the solution and forms small dispersed regions of liquid crystal in a polymer matrix. The oligomer may be non-polar or comprise non-polar substituents to increase the miscibility with the liquid crystal. Suitably, as aa monomer is smaller than an oligomer, a monomer is preferred where solubility with the liquid crystal is desired.

Advantageously, the liquid crystal is finely dispersed in the continuous phase and the viscous structure of the gel reduces reaggregation of the liquid crystal. Curing of the continuous phase comprising the oligomer and/or monomer then fixes the dispersed regions of liquid crystal in place thereby providing excellent colour change characteristics

Suitably, the curable oligomer comprises a urethane. Preferably the urethane oligomer is selected from aliphatic urethane acrylates, aliphatic urethane methacrylates, polyether urethane acrylates, polyether urethane methacrylates, polyester urethane acrylates and polyester urethane methacrylates.

The composition of the invention may comprise a curable monomer. Suitably, the monomer is polar and immiscible with the liquid crystal. The monomer may comprise shellac. Preferably the monomer comprises a hydroxyl-substituted alkyl acrylate or alkyl methacrylate, for example hydroxyethylmethacrylate. Examples of suitable monomers include isobornyl methacrylate, 2-hydroxyethyl methacrylate.

In one embodiment, the monomer may be difunctional or multi-functional so as to facilitate formation of cross-links which may impart improved hardness and/or abrasion resistance to the coating. An example of a suitable difunctional monomer includes. di-HEMA trimethylhexyl dicarbamate.

In another embodiment, the monomer comprises shellac and the coating composition is substantially free of an oligomer and is not in gel form.

The monomer may also act as a viscosity modifier to provide improved thixotropic (shear-thinning) properties and aid wetting. Advantageously, the composition may be thicker when stored and readily thinned upon application aiding storage stability improving performance upon application. In one embodiment, the continuous phase comprises an oligomer and monomer is added to the oligomer to reduce its viscosity to a desired level.

The curable oligomer and/or monomer is suitably present in the coating composition at a level of 90 to 10%, preferably 80 to 25%, more preferably 70 to 35%, especially 65 to 50% by weight. Preferably, a monomer is present in combination with the oligomer. Preferably the monomer is present at a level of 1 to 30%, more preferably 5 to 20% especially 5 to 15% by weight of the composition. In an especially preferred example, the oligomer is present at a level of 35 to 70% and the monomer is present at a level of 5 to 15%.

The oligomer and/or monomer may be dissolved or dispersed in a non-aqueous solvent or may simply be mixed together with the initiator and unsealed liquid crystal without inclusion of a solvent.

Suitably, the oligomer and/or monomer are cured using a UV source for example a broad spectrum, lower intensity lamp, or a LED source, for example a LED lamp with narrow bandwidth and high intensity. Suitably, the coating is cured within 30 seconds to 5 minutes, preferably 1 to 3 minutes per coating with a UV source and less than 1 minute for an LED source.

The coating composition according to the invention may be applied directly to the nail or may be applied to an undercoat which has been applied to the nail. The undercoat suitably comprises a solvent free gel composition. Any compositions commercially available in the field may be employed for example NAILFX base coat gel by the Edge nails, and one step UV LED gel nail polish by Lazy Nails

The coating composition according to the invention, once applied to the nail may be coated with an overcoat. The overcoat may provide an aesthetic or functional purpose or both, for example providing a protective coating for the coating composition. The overcoat suitably comprises a solvent free gel coat known in the art and any compositions commercially available in the field may be employed for example SUPERNAIL by Progel

In a further aspect, the invention provides the use of a chromic product comprising a liquid crystal, preferably a polymer dispersed liquid crystal in a coating comprising a continuous polymer matrix derived from a curable oligomer and/or a curable monomer to provide a colour-change effect, preferably an intense colour change, on a substrate, preferably a human or animal nail, hoof or the like.

The coating may be applied to a substrate by conventionally using a brush. Additionally, the gel may be applied into a mould before being placed onto the nail and exposed to UV radiation or heat, upon which the nail will cure.

The cured coating composition may be non-iridescent or iridescent according to the desired effect. Other appearances may be provided, for example pearlescent, matt, gloss and fluorescent, particularly with incident light near the UV spectrum. Iridescence suitably provides scattering of incident light to provide a shimmering effect of multiple colours or a rainbow of colours and arises due to the nail product having suitable refractive characteristics. Advantageously, PDLC liquid crystals may provide iridescent qualities as well as colour-change properties.

The coating preferably comprises an abrasion additive to provide hardness and resistance to abrasion for the product once coated on the substrate. Suitably, the abrasion additive is selected from an oxidised polyethylene wax or any other suitable abrasion additive-.

The coating may comprise an optical brightener to promote colour intensity. Any known optical brighteners may be employed, for example stilbenes.

Optionally, the coating may comprise a rheology modifier to aid dispersibility and spreading. The rheology modifier may be inorganic or organic. Examples of suitable rheology modifiers include a natural thickener, a synthetic thickener and/or a dispersant or surfactant. Any known thickeners or surfactants suitable for use in the intended field of use of the composition may be employed. Preferably, the rheology modifier is at most partially soluble in water and desirably not water soluble.

Examples of organic thickeners include alkali-swellable emulsions (ASEs), hydrophobically modified alkali-swellable emulsions (HASEs) and hydrophobically ethoxylated urethane resins (HEURs). Examples of inorganic rheology modifiers include clays and silicates such as bentonite, magnesium aluminium silicate, modified bentonite, and other mineral rheology modifiers.

The coating composition may comprise a UV additive. The UV additive may be soluble in oil or water but preferably is insoluble in water. Suitable UV additives include benzophenone-4 and phenylbenzimidazole sulfonic acid (EUSOLEX 232 available from Merck). The coating composition may comprise a UV stabiliser, however this may hinder the action of the photoinitiator. More preferably, the UV absorber/stabiliser will be included in a top coat to be added on top of the liquid crystal-containing layer once cured. This top coat layer may be cured by intense LED lamps in order to take advantage of the near-visible light that will not be as strongly hindered by the UV absorber/stabiliser in the top coat. Suitable examples of photoinitiators which could be used in such a top coat include SPEEDCURE TPO-L supplied by Lambson.

The UV absorber/stabiliser will be either a chemical or physical/mineral absorber, more preferably a chemical UV stabiliser, as a mineral absorber will cover/drown out the colour effect of the liquid crystal. Suitable examples of UV absorbers which can be included in the top coat include avobenzone, octyl methoxycinnamate etc.

The invention also provides a cured coating, preferably a cured gel coating, according to the invention coated on a nail to provide a chromic layer and a top-coat comprising a cured oligomer and/or monomer and a UV absorber coated on the chromic layer

The coating composition may also comprise a plasticiser, with known commercially available plasticisers being suitable.

The coating composition may also comprise a perfume, especially if the composition is intended for use on the human or animal body. Examples of suitable perfumes include peppermint oil, sweet orange essential oil, menthol, linalool, parfum, methyl butyrate, citral, myrcene, limonene and eucalyptol.

As desired, the coating composition may comprise a colourant in addition to the colour-change colourant.

Examples of suitable dyes include any dyes suitable for use in cosmetic products may be employed including the following used in combination or on their own; N,N-Bis(2-Hydroxyethyl)-2-Nitro-p-Phenylenediamine, 4-Amino-3-Nitrophenol, 4-Hydroxypropylamino-3-Nitrophenol, Hydroxyethyl-2-nitro-p-toluidine, HC Blue No. 12, 3-Nitro-p-Hydroxyethylaminophenol, 2-Amino-6-Chloro-4-Nitrophenol, Acid Red 33, HC Yellow No. 2, HC ORANGE NO. 1, HC YELLOW NO. 13, Basic Yellow 57, HC Red No. 3, Basic Red 76, Basic Red 51, Acid Red 92, Basic Yellow 87, Basic Orange 31, BASIC VIOLET 2, HC Blue No. 16, Acid Violet 43, Basic Brown 17 and dyes provided by Sensient® Cosmetic technologies; Arianor Jade Blue (HC Blue 15), phat black DC 9206 (mixture of C161565, C160725, basic brown 16, acid violet 43, basic red 76, CI 26100), Arianor flash deep black (Basic Blue 99, Basic Brown 16, Acid Violet 43, Basic Red 76, Basic Yellow 57, Basic Brown 17, Polyquaternium-37, Hydrolyzed Yeast Protein)

Examples of suitable pigments include any pigments suitable for use in cosmetic products may be employed including the following used in combination or on their own; Iron oxides, titanium oxides, synthetic mica (synthetic fluorphlogopite), carbon black, lakes, natural mica, silica, Covalumine fire red AS (CI 15850, Alumina, Triethoxycaprylylsilane), Noir W 699 (CI 20470, Sodium Sulfate), Covalumine astral blue (CI 42090, Alumina, Triethoxycaprylylsilane), Unipure white LC986 FSP (CI 77891, Perfluorooctyl Triethoxysilane, Polyperfluoromethylisopropyl Ether), Unipure red LC3071 (CI 15850, Aluminum Hydroxide), Unipure white LC987 GCA (CI 77891, Sodium Cocoyl Glutamate, Cystine, Lauroyl Arginine), Covarine white WN9787 (CI 77891, Aqua, Glycerin, Xanthan Gum, Sodium Citrate), Covapate Uniwhite LC 9781 (Ricinus communis (Castor) Seed Oil, CI 77891, Polyhydroxystearic Acid), Unipure white LC981 SGP (CI 77891, Sodium Glycerophosphate), Vert covasol W7035 (CI 19140, CI 42090), unipure yellow LC182 ADT-C(CI 77492, Isopropyl Titanium Triisostearate, Bis-PEG-15 Dimethicone/IPDI Copolymer, PEG-2 Soyamine), unipure black LC989 ADT-C(CI 77499, Isopropyl Titanium Triisostearate, Bis-PEG-15 Dimethicone/IPDI Copolymer, PEG-2 Soyamine), unipure black LC988 FSP (CI 77499, Perfluorooctyl Triethoxysilane, Polyperfluoromethylisopropyl Ether).

The invention is illustrated by the following non-limiting examples. Percentage and part quantities are by weight based on the composition of the invention unless otherwise stated.

### EXAMPLES

### Example 1

A composition was prepared by mixing the following components in the amounts (by weight based on the whole composition) specified in the following tables.

| Component | Percentage (%) |
|---|---|
| BR-541 (polyether urethane acrylates, supplied | 50 |
| by Dymax) | |
| Unsealed cholesteric liquid crystal | 40 |
| HEMA (hydroxyethyl methacrylate) | 5 |
| TPO-L (Ethyl (2,4,6-trimethylbenzoyl) phenyl | 5 |
| phosphinate | |

### Example 2

A composition was prepared by mixing the following components in the amounts (by weight based on the whole composition) specified

| Component | Percentage (%) |
|---|---|
| Genomer 4247 (aliphatic urethane | 35 |
| methacrylate) | |
| Urethane acrylate 03-978 | 10 |
| 1121M (isobornyl methacrylate) | 2 |
| TPO-L (Ethyl (2,4,6-trimethylbenzoyl) phenyl | 2 |
| phosphinate | |
| Unencapsulated cholesteric liquid crystal | 50 |

### Example 3

A UV top coat formulation having the following components was prepared:

| Component | Percentage (%) |
|---|---|
| Genomer 4247 (aliphatic urethane | 68 |
| methacrylate) | |
| Urethane acrylate 03-978 | 20 |
| 1121M (isobornyl methacrylate) | 5 |
| TPO-L (Ethyl (2,4,6-trimethylbenzoyl) phenyl | 5 |
| phosphinate | |
| Avobenzone | 2 |

The top coat formulation was applied to a nail coated with a gel coating according to the invention as set out in Examples 1 and 2 and provided improved durability, resistance to abrasion and UV protection.

### Example 4

A black base coat was painted onto a nail. A gel composition according to Example 1 was then applied. Upon curing, the gel coating provided attractive colour change effect and a durable finish. This procedure was repeated with a gel composition according to Example 2. An attractive durable finish was obtained.

1. A coating composition comprising a chromic component comprising a liquid crystal at a level of 30 to 80% by weight of the composition, and a curable oligomer and/or, a monomer and a curing initiator.

2. A coating composition according to claim 1 wherein the oligomer and/or monomer is dissolved or dispersed in a non-aqueous solvent.

3. A coating composition according to claim 1 wherein the liquid crystal comprises a cholesteric liquid crystal and/or a phenyl benzoate esters.

4. A coating composition according to claim 1 wherein the liquid crystal is present at a level of 35 to 75% by weight of the composition.

5. A coating composition according to claim 1 comprising a colourant.

6. A coating composition according to claim 5 wherein the colourant is selected from a static liquid crystal, a dye and a pigment.

7. A coating composition according to claim 5 wherein the colourant comprises a powder or a non-aqueous solution.

8. A coating composition according to claim 1 comprising a chromic component comprising an unsealed liquid crystal, a curable oligomer and/or a monomer and an initiator and a colourant added to the composition in powder form.

9. A coating composition according to claim 1 wherein the curable oligomer and/or curable monomer are polar and immiscible with the liquid crystal.

10. A coating composition according to claim 1 comprising a curable oligomer comprising a urethane.

11. A coating composition according to claim 1 comprising a curable oligomer selected from one or more of aliphatic urethane acrylates, aliphatic urethane methacrylates, polyether urethane acrylates, polyether urethane methacrylates, polyester urethane acrylates and polyester urethane methacrylates.

12. A coating composition according to claim 1 comprising a curable monomer selected from one or more of a hydroxyl-substituted alkyl acrylate or alkyl methacrylate, and isobornyl methacrylate

13. (canceled)

14. A coating composition according to claim 1 comprising the oligomer and a monomer wherein the oligomer is present at a level of 35 to 70% and the monomer is present at a level of 5 to 15%.

15. A coating product comprising as separate compositions, a coating composition according to claim 1 and a non-aqueous composition comprising a curable oligomer and./or monomer and a UV absorber.

16. (canceled)

17. A coating composition according to claim 1 adapted to coat nails.

18. (canceled)

19. A coating composition according to claim 1 wherein the coating composition is a gel coating composition.

20. A cured coating comprising a chromic component comprising a liquid crystal at a level of 30 to 80% by weight of the coating and a continuous polymer phase which is derived from a cured oligomer and/or, a cured monomer, and an initiator, wherein the chromic component is dispersed in the said continuous phase.

21. A cured coating according to claim 20 coated on a nail to provide a chromic layer and a top-coat comprising a cured oligomer and/or monomer and a UV absorber coated on the chromic layer.

22. A method of providing a cured coating which comprises applying a coating composition to a substrate to be coated which coating composition comprises a chromic component comprising a liquid crystal at a level of 30 to 80% by weight of the composition, and a curable oligomer and/or a monomer and an initiator, curing the curable oligomer and/or monomer to form a polymer to provide a continuous phase comprising the polymer and a disperse phase comprising the chromic composition dispersed in the continuous phase.

23. (canceled)

24. A process of providing a colour-change effect on a keratinous substrate comprising applying a chromic product comprising a liquid crystal coating comprising a continuous polymer matrix derived from a curable oligomer and/or a curable monomer to provide a colour-change effect to a keratinous substrate.
